SEQUENCE LISTING (1 sequence)

SEQ ID NO: 1 - DNA, 20 bp, Artificial Sequence
  EPS8L1 siRNA
aacagcctccgtgcttagca

(19) United States
(12) Patent Application Publication
(10) Pub. No.: US 2016/0273045 A1
(43) Pub. Date: Sep. 22, 2016

(54) METHODS OF DETERMINING BREAST CANCER PROGNOSIS

(71) Applicant: OREGON HEALTH & SCIENCE UNIVERSITY, Portland, OR (US)

(72) Inventors: Juha Rantala, Turku (FI); Joe W. Gray, Lake Oswego, OR (US)

(73) Assignee: Oregon Health & Science University, Portland, OR (US)

(21) Appl. No.: 15/031,550

(22) PCT Filed: Oct. 23, 2014
(86) PCT No.: PCT/US2014/061981

§ 371 Date: Apr. 22, 2016

Related U.S. Application Data
(60) Provisional application No. 61/894,548, filed on Oct. 23, 2013.

(51) Int. Cl. C12Q 1/68; G01N 33/574

(52) U.S. Cl. CPC C12Q 1/6886; G01N 33/57415; C12Q 2600/118; C12Q 2600/156; C12Q 2600/158; G01N 2333/912

(57) ABSTRACT

Disclosed herein are methods of determining a diagnosis or prognosis for a subject with a breast tumor. In one embodiment, the method includes determining an amount of EPS8-like 1 (EPS8L1) in the sample (such as an amount of EPS8L1 nucleic acid or protein) and comparing the amount of EPS8L1 in the sample to a control. The subject is determined to have a poor prognosis (such as decreased likelihood of survival) if the amount of EPS8L1 in the sample is increased compared to the control. In some embodiments, the methods further include administering a treatment to a subject determined to have a poor prognosis, such as administering an ErbB2-targeting therapy to the subject.

### CROSS-REFERENCE TO RELATED APPLICATION

This application claims the benefit of the earlier filing date of U.S. Provisional Patent Application No. 61/894,548, filed on Oct. 23, 2013, which is incorporated herein by reference in its entirety.

### ACKNOWLEDGMENT OF GOVERNMENT SUPPORT

This invention was made with government support under grant number U54CA112970 awarded by the National Institutes of Health. The government has certain rights in the invention.

### FIELD

This disclosure relates to a genetic marker for ErbB2-positive breast cancer and methods for determining a diagnosis and/or prognosis of ErbB2-positive breast cancer.

### BACKGROUND

Breast cancer is the most common cancer in women worldwide and is the most common cause of death from cancer in women worldwide. However, breast cancer is a heterogeneous disease and varies widely in response to standard therapies. Identification of molecular variation among breast cancers has led to improved prognosis and treatment for patients. For example, the identification of amplification and/or overexpression of ErbB2 (Her2) in many breast tumors has resulted in treatment of patients with ErbB2 positive tumors with ErbB2-targeting therapies (such as trastuzumab and/or lapatinib). In many cases, these therapies are effective; however, some ErbB2 positive tumors do not respond to treatment or become resistant to ErbB2-targeting therapies. Thus, there remains a need for further molecular characterization and stratification of breast tumors for providing improved diagnosis, prognosis, and/or treatment options for patients.

### SUMMARY

Disclosed herein are methods of determining a diagnosis or prognosis for a subject with a breast tumor. In some examples, determining the diagnosis includes determining whether a tumor is benign or malignant. In other examples, determining the prognosis includes predicting the outcome (for example, likelihood of survival) of a subject with a breast tumor. In one embodiment, the method includes determining an amount of EPS8-like 1 (EPS8L1) nucleic acid and/or protein in a sample (such as a breast tumor sample) from a subject and comparing the amount of EPS8L1 nucleic acid and/or protein in the sample to a control. The subject is determined to have a poor prognosis (such as decreased likelihood of survival) if the amount of EPS8L1 in the sample is increased compared to the control.

In some embodiments, the disclosed methods further include determining an amount of ErbB2 nucleic acid or protein in the sample from the subject. In some examples, subjects with increased ErbB2 nucleic acid or protein (such as increased ErbB2 mRNA, protein, gene copy number and/or gene amplification) in the sample in addition to increased EPS8L1 nucleic acid or protein have a particularly poor outcome.

In additional embodiments, the methods further include administering a treatment to a subject determined to have a poor prognosis, such as administering an ErbB2-targeting therapy to the subject.

The foregoing and other features of the disclosure will become more apparent from the following detailed description, which proceeds with reference to the accompanying figures.

### BRIEF DESCRIPTION OF THE DRAWINGS

At least some of the following figures are submitted in color.

FIG. 1A is a heatmap showing identification of siRNA that caused growth inhibitory and apoptotic responses when contacted with ErbB2 positive breast cancer cell lines.

FIG. 1B is a plot showing expression of EPS8L1 in six selected cell lines relative to other genes. Four genes (STARD3, ERBB2, DOCK9, and RAB20) are indicated.

FIG. 2A is a bar graph showing the frequency of genomic aberrations of EPS8L1 in the indicated solid tumors from The Cancer Genome Atlas. At least 4% of all invasive breast carcinomas have an EPS8L1 alteration.

FIG. 2B is a bar graph showing the mRNA expression pattern of EPS8L1 across a panel of human breast cancer cell lines (median centered log 2). Cell lines are grouped in the basal A (red, left), basal B (grey, center) and luminal (blue, right) subgroups.

FIG. 2C is a box and whiskers plot of the stratification of EPS8L1 mRNA expression according to the indicated molecular subtype.

FIG. 3A is a set of four plots indicating a time lapse growth assay for two different EPS8L1 siRNAs (center right and far right panels) as well as negative (far left) and positive (center left) controls.

FIG. 3B is an image of a western blot of EPS8L1 expression in the presence of siRNA or controls as indicated.

FIG. 3C is an image of a western blot showing EPS8L1 and ErbB2 in the indicated cell lines.

FIG. 4A is a Kaplan-Meier plot of overall survival (OS) times based on EPS8L1 expression of all breast cancer tumors.

FIG. 4B is a Kaplan-Meier plot of overall survival (OS) times based on EPS8L1 expression of Her2 (ErbB2)-enriched tumors.

FIG. 4C is a Kaplan-Meier plot of overall survival (OS) times based on EPS8L1 expression of ER-positive tumors.

FIG. 4D is a Kaplan-Meier plot overall survival (OS) times based on EPS8L1 expression of ER-negative, Her2-enriched tumors.

FIG. 5A is a Kaplan-Meier plot of cumulative survival times based on EPS8L1 and ErbB2 copy number with DNA gain and amplification separated.

FIG. 5B is a Kaplan-Meier plot of cumulative survival times based on EPS8L1 and ErbB2 copy number with DNA gain and amplification combined.

### SEQUENCE LISTING

Any nucleic acid and amino acid sequences listed herein or in the accompanying sequence listing are shown using standard letter abbreviations for nucleotide bases and amino acids, as defined in 37 C.F.R. §1.822. In at least some cases, only one strand of each nucleic acid sequence is shown, but the complementary strand is understood as included by any reference to the displayed strand.

SEQ ID NO: 1 is an exemplary EPS8L1 RNAi nucleic acid sequence.

### DETAILED DESCRIPTION

It is disclosed herein that EPS8L1 is a marker useful for detecting particularly aggressive advancing forms of ErbB2-positive breast cancer. The present disclosure provides methods of improved accuracy for the diagnosis and prognosis of ErbB2-positive breast cancer by stratifying this subtype of breast cancer into further sub-classes. In the current context of clinical stratification of ErbB2-positive breast cancer, standard procedures to determine molecular subtype of the tumor are generally based on pathological grading and a limited set of molecular markers, in which protein level expression of ErbB2 is analyzed alone or in combination with detection of DNA copy number gain of the ErbB2 genomic locus. The data obtained in such experiments, together with current assumptions about molecular characterization of breast cancers is used to estimate a theoretical clinical behavior for the tumor cell population. However, these approaches will underestimate intra-tumor complexity, such as molecular heterogeneity of cancerous cells, for example if a considerable fraction of the cells can undergo differentiation changes and thus are missed by the single marker labeling. In contrast, EPS8L1 directly reflects a certain physiological state of the cell, as EPS8L1 marks pathway dependency of the cells to signaling through ErbB-family receptors in breast tissue. Although the functional role of EPS8L1 is not yet fully understood, it is clear that EPS8L1 protein expression and cell proliferation are closely linked.

The disclosed methods include identifying EPS8L1 and determining the expression or gene copy number of EPS8L1 in a sample from a subject (such as a subject with breast cancer) and comparing this to a control (such as EPS8L1 expression or copy number in a sample from a healthy or unaffected individual). A difference in expression or gene copy number of EPS8L1 indicates that the subject has an increased risk of dying of aggressively progressing breast cancer initially classified only as ErbB2-positive. Thus, in some examples, the disclosed methods also include identifying whether the sample from the subject is ErbB2-positive (for example, has an increased amount of ErbB2 expression or copy number as compared to a control).

### I. TERMS

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. All sequences associated with the GenBank Accession Nos. mentioned herein are incorporated by reference in their entirety as were present on Oct. 7, 2013, to the extent permissible by applicable rules and/or law. In case of conflict, the present specification, including explanations of terms, will control. In addition, the materials, methods, and examples are illustrative only and not intended to be limiting.

In order to facilitate review of the various embodiments of this disclosure, the following explanations of specific terms are provided:

Cancer: A malignant neoplasm that has undergone anaplasia with loss of differentiation, increased rate of growth, invasion of surrounding tissue, and is capable of metastasis. For example, breast cancer is a malignant neoplasm that arises in or from breast tissue (such as a ductal carcinoma). Breast cancers are frequently classified as luminal A (ER positive and/or PR positive, ErbB2 negative, and low Ki67), luminal B (ER positive and/or PR positive and ErbB2 positive, or ErbB2 negative with high Ki67), basal-like or triple-negative (ER negative, PR negative ErbB2 negative, cytokeratin 5/6 positive and/or HER1 positive), or ErbB2 positive (ER negative, PR negative, ErbB2 positive). However, breast cancers may be heterogeneous both between individuals and at the cellular level within a tumor, and one of skill in the art will understand that they may not always fit within the classification scheme.

Residual cancer is cancer that remains in a subject after any form of treatment is given to the subject to reduce or eradicate cancer. Metastatic cancer is a cancer at one or more sites in the body other than the original site of the cancer from which the metastatic cancer is derived. Local recurrence is a reoccurrence of the cancer at or near the same site as the original cancer, for example, in the same tissue as the original cancer.

Control: A sample or standard used for comparison with an experimental sample. In some embodiments, the control is a sample obtained from a healthy patient or a non-tumor tissue sample obtained from a patient diagnosed with cancer. In other embodiments, the control is a historical control or standard reference value or range of values (such as a previously tested control sample, such as a group of cancer patients with known prognosis or outcome, or group of samples that represent baseline or normal values, such as the level of EPS8L1 in non-tumor tissue). In other examples, a control is a threshold value.

EPS8L1: EPS8-like 1; also known as epidermal growth factor receptor kinase substrate 8-like protein 1, EPS8-related protein 1. EPS8L1 is related to a substrate for the epidermal growth factor receptor (epidermal growth factor receptor pathway. The function of EPS8L1 is unknown.

Nucleic acid and amino acid sequences for EPS8L1 are publicly available. For example, EPS8L1 genomic DNA is disclosed in GenBank Accession No. NC_000019.9 (nucleotides 55587221-55599291), incorporated by reference as provided in GenBank on Oct. 7, 2013. In addition, GenBank Accession Nos. NM_133180, NM_017729, NM_139204, XM_005259020, XM_005259021, and XM_005259022 disclose exemplary human EPS8L1 nucleic acid sequences, and GenBank Accession Nos. NP_573441, NP_060199, NP_631943, XP_005259077, XP_005259078, XP_05059079 disclose exemplary human EPS8L1 protein sequences, all of which are incorporated by reference as provided in GenBank on Oct. 7, 2013. One of ordinary skill in the art can identify additional EPS8L1 sequences and variants thereof.

ErbB2: Also known as v-erb-b2 avian erythroblastic leukemia viral oncogene homolog 2, c-erbB2/neu, her2/neu, or Her2. ErbB2 is a member of the epidermal growth factor receptor family of tyrosine kinases. It is amplified and/or overexpressed in several cancers, including breast and ovarian cancer. ErbB2 does not have a ligand binding domain and cannot bind ligands itself. However, ErbB2 heterodimerizes with other members of the EGF receptor family, stabilizing ligand binding and kinase-mediated activation of intracellular signaling pathways.

ErbB2 nucleic acid and protein sequences are publicly available. For example, ErbB2 genomic DNA is disclosed at GenBank Accession No. NC_000017.10 (nucleotides 37844167-37884915), incorporated by reference as provided in GenBank on Oct. 7, 2013. In addition, GenBank Accession Nos. XM_005257139, NM_001005862, NM_004448, and XM_005257140 disclose exemplary human ErbB2 nucleic acid sequences and GenBank Accession Nos. XP_005257196, NP_001005862, NP_004439, and XP_005257197 disclose exemplary human ErbB2 amino acid sequences, all of which are incorporated herein by reference as present in GenBank on Oct. 7, 2013. One of ordinary skill in the art can identify additional ErbB2 sequences and variants thereof.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

In vitro determination: Determining a value or amount by using laboratory techniques that require the transformation of a sample (such as a tissue sample) in the laboratory, for example by reaction with reagents, such as antibodies, nucleic acids, and/or labels that identify one or more targets within the sample. For example, in vitro determination can indicate whether a target is increased or decreased in a sample relative to a control. An in vitro determination requires more than the manipulation of abstract information.

Label (or detectable label): An agent capable of detection, for example by ELISA, spectrophotometry, flow cytometry, or microscopy. For example, a label can be attached to a nucleic acid molecule or protein (such as a probe or antibody), thereby permitting detection of a target nucleic acid molecule or protein. Examples of labels include, but are not limited to, radioactive isotopes, enzyme substrates, co-factors, ligands, chemiluminescent agents, fluorophores, haptens, enzymes, and combinations thereof. In other examples, the labels are synthetic (non-naturally occurring) labels. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Oligonucleotide probes and primers: A probe includes an isolated nucleic acid attached to a detectable label or reporter molecule. Primers are short nucleic acids, preferably DNA oligonucleotides, of about 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example by polymerase chain reaction (PCR) or other nucleic-acid amplification methods known in the art. One of skill in the art will appreciate that the specificity of a particular probe or primer increases with its length. Thus, for example, a probe or primer comprising 20 consecutive nucleotides will anneal to a target with a higher specificity than a corresponding probe or primer of only 15 nucleotides. Thus, in order to obtain greater specificity, probes and primers can be selected that comprise about 20, 25, 30, 35, 40, 50 or more consecutive nucleotides.

Prognosis: Prediction of the course of a disease, such as cancer (for example, breast cancer). The prediction can include determining the likelihood of a subject to develop aggressive, recurrent disease, to develop one or more metastases, to survive a particular amount of time (e.g., determine the likelihood that a subject will survive 1, 2, 3, 5, 10 years or more), to respond to a particular therapy, or combinations thereof. The prediction can also include determining whether a tumor is a malignant or a benign tumor.

Sample (or biological sample): A biological specimen containing DNA, RNA (including mRNA), protein, or combinations thereof, obtained from a subject. Examples include, but are not limited to, peripheral blood, urine, saliva, tissue biopsy, fine needle aspirate, surgical specimen, and autopsy material. In some examples, a sample includes a tumor sample, such as a fresh, frozen, or fixed tumor sample, for example a formalin-fixed paraffin-embedded tumor sample.

Subject: Living multi-cellular vertebrate organisms, a category that includes human and non-human mammals, such as veterinary subjects.

Survival: Time interval between date of diagnosis or first treatment (such as surgery or first chemotherapy) and a specified event, such as relapse, metastasis or death. Overall survival is the time interval between the date of diagnosis or first treatment and date of death or date of last follow up. Relapse-free survival is the time interval between the date of diagnosis or first treatment and date of a diagnosed relapse (such as a locoregional recurrence) or date of last follow up. Metastasis-free survival is the time interval between the date of diagnosis or first treatment and the date of diagnosis of a metastasis or date of last follow up.

Tumor: The product of neoplasia is a neoplasm (a tumor), which is an abnormal growth of tissue that results from excessive cell division. A tumor that does not invade surrounding tissue or metastasize is referred to as “benign.” A tumor that invades the surrounding tissue and/or can metastasize is referred to as “malignant.” In some examples, a tumor is a breast tumor.

### II. OVERVIEW OF SEVERAL EMBODIMENTS

Disclosed herein are methods of determining a diagnosis or prognosis for a subject with a breast tumor. In some examples, determining the diagnosis includes determining whether a tumor is benign or malignant. In other examples, determining the prognosis includes predicting the outcome (for example, likelihood of survival) of a subject with a breast tumor. In one embodiment, the method includes determining an amount of EPS8L1 (such as an amount of EPS8L1 nucleic acid or protein) in a sample (such as a tumor sample, for example, a breast tumor sample) from a subject with a breast tumor and comparing the amount of EPS8L1 in the sample to a control. The subject is determined to have a poor prognosis (such as decreased likelihood of survival) if the amount of EPS8L1 in the breast tumor sample is increased compared to the control. In other examples, the methods include determining that a subject has breast cancer if the amount of EPS8L1 in the breast tumor sample is increased compared to the control. The disclosed methods may also be used to determine a diagnosis or prognosis for a subject with any type of tumor that has increased EPS8L1 nucleic acid and/or protein, for example an ovarian tumor.

In some examples, the methods include determining an amount of an EPS8L1 nucleic acid in a breast tumor sample. The EPS8L1 nucleic acid can include genomic DNA (for example, determining EPS8L1 gene copy number or the presence of gene amplification in the sample) or mRNA or cDNA (for example, determining expression of EPS8L1 in the sample). In other examples, the methods include determining an amount of an EPS8L1 protein in a breast tumor sample. In some embodiments, the methods further include detecting one or more additional nucleic acids or proteins in the breast tumor sample, including but not limited to ErbB2, estrogen receptor, progesterone receptor, and Ki67. In particular examples, the methods include determining EPS8L1 gene copy number, presence of EPS8L1 gene amplification and/or amount of EPS8L1 mRNA or protein in the sample and comparing the amount of EPS8L1 nucleic acid or protein with a control and determining ErbB2 gene copy number, presence of ErbB2 gene amplification, and/or amount of ErbB2 mRNA or protein in the sample and comparing the amount of ErbB2 nucleic acid or protein with a control. Subjects with a combination of increased EPS8L1 nucleic acid and/or protein and increased ErbB2 nucleic acid and/or protein have a particularly poor prognosis. In some examples, subjects with EPS8L1 copy number gain and ErbB2 amplification have an average survival time of less than 48 months (such as less than 42 months, less than 36 months, less than 30 months, less than 24 months, less than 18 months, less than 12 months, less than 6 months, or less than 3 months). In other examples, subjects with EPS8L1 copy number loss and ErbB2 copy number gain have a particularly good prognosis (such as an average survival time of more than 5 years, more than 7 years, more than 10 years, more than 12 years, more than 15 years, or even longer). Methods of determining an amount of a nucleic acid or protein in a sample are known to one of ordinary skill in the art and are discussed in more detail below.

In some embodiments, the disclosed methods utilize a sample from a patient with a breast tumor. In other embodiments, the methods utilize a sample from a patient with a tumor having or suspected of having increased EPS8L1 nucleic acid and/or protein (for example, a patient with an ovarian tumor). In some examples, the sample includes tumor cells, for example, a tumor sample (such as a breast tumor sample). The sample may also include non-tumor cells, for example, adjacent to or intermingled with the tumor cells. In particular examples, the sample includes a tissue, biopsy, or bodily fluid from the subject (such as a breast tumor biopsy or a fine needle aspirate). In some examples, a sample includes a tumor sample, such as a fresh, frozen, or fixed tumor sample, for example a formalin-fixed paraffin-embedded tumor sample. In other examples, the sample includes circulating tumor cells (such as a blood sample or a sample including at least one fraction of a blood sample). In additional examples, the sample can include isolated nucleic acids (such as DNA, RNA, mRNA, or cDNA) or protein from a sample including tumor cells.

Poor prognosis can refer to any negative clinical outcome, such as, but not limited to, a decrease in likelihood of survival (such as overall survival, relapse-free survival, or metastasis-free survival), a decrease in the time of survival (e.g., a predicted average survival of less than 10 years, less than 5 years, less than 3 years, less than 2 years, or less than one year), presence of a malignant tumor, an increase in the severity of disease, a decrease in response to therapy, an increase in tumor recurrence, an increase in metastasis, or the like. In particular examples, a poor prognosis is a decreased chance of survival (for example, a predicted average survival time of equal to or less than 10 years, such as less than 9 years, 8 years, 7 years, 6 years, 5 years, 4 years, 3 years, 24 months, 18 months, 12 months, 6 months, or 3 months from time of diagnosis or first treatment).

In some embodiments of the method, an alteration in the amount of EPS8L1 nucleic acid and/or protein in the sample relative to a control indicates a poor prognosis. In some examples, an increase (such as a statistically significant increase) in amount of EPS8L1 gene copy number and/or EPS8L1 mRNA and/or protein relative to the control indicates a poor prognosis. For example, an increase in the amount of EPS8L1 nucleic acid and/or protein relative to a control or reference value (or range of values) indicates a poor prognosis, such as a decreased chance of survival (for example decreased overall survival, relapse-free survival, or metastasis-free survival). In some examples, a decreased chance of survival includes a predicted average survival time of equal to or less than 50 months, such as less than 48 months, 42 months, 36 months, 30 months, 24 months, 18 months, 12 months, 9 months, 6 months, or 3 months from time of diagnosis or first treatment. In other examples, no significant change, or a decrease, in the amount of EPS8L1 nucleic acid and/or protein relative to the control indicates a good prognosis (such as increased chance of survival, for example increased overall survival, relapse-free survival, or metastasis-free survival). In a specific example, no significant change, or a decrease in amount of EPS8L1 nucleic acid and/or protein relative to the control indicates a good prognosis such as an increased chance of survival for example, a predicted average survival time of at least 50 months, such as at least 5 years, at least 6 years, at least 7 years, at least 8 years, at least 9 years, at least 10 years, at least 12 years, or more from time of diagnosis or first treatment.

In additional embodiments, an increase in the amount of EPS8L1 nucleic acid and/or protein relative to a control (such as an increase in EPS8L1 gene copy number or EPS8L1 gene amplification) and an increase in the amount of ErbB2 nucleic acid and/or protein relative to a control (such as an increase in ErbB2 gene copy number or ErbB2 gene amplification) indicates a poor prognosis, such as a decreased chance of survival. In some examples, the decreased chance of survival includes a survival time of equal to or less than 50 months, such as less than 48 months, 42 months, 36 months, 30 months, 24 months, 18 months, 12 months, 9 months, 6 months, or 3 months from time of diagnosis or first treatment. In other examples, a decrease in the amount of EPS8L1 nucleic acid and/or protein in the sample relative to a control (such as a loss of EPS8L1 nucleic acid) and an increase in ErbB2 nucleic acid and/or protein (such as ErbB2 gain) in the sample relative to a control indicates a good prognosis, such as an increased chance of survival. In some examples, the increased chance of survival includes a survival time of equal to or greater than at least 50 months, such as at least 5 years, at least 6 years, at least 7 years, at least 8 years, at least 9 years, at least 10 years, at least 12 years, or more from time of diagnosis or first treatment.

In particular examples, an amount of EPS8L1 nucleic acid or protein in the sample at least 1.25-fold greater than a control (such as at least 1.5-fold greater, at least 2-fold greater, at least 2.5-fold greater, at least 3-fold greater, at least 4-fold greater, at least 5-fold greater, at least 10-fold greater, or more as compared to a control) indicates that the subject has a poor prognosis. In other examples, presence of an increased gene copy number or gene amplification in the sample indicates that the subject has a poor prognosis. In some examples, an EPS8L1 gene copy number greater than 2 (such as greater than about 2, 3, 4, 5, 10, 20, or more) or a ratio of EPS8L1 gene copy number to Chromosome 19 copy number greater than about two (such as greater than about 2, 3, 4, 5, 10, 20, or more) indicates a poor prognosis for the subject.

The control can be any suitable control against which to compare an amount of an EPS8L1 nucleic acid or protein in a tumor sample. In some embodiments, the control sample is non-tumor tissue. In some examples, the non-tumor tissue is obtained from the same subject, such as non-tumor tissue that is adjacent to the tumor. In other examples, the non-tumor tissue is obtained from a healthy control subject. In other embodiments, the control is a reference value or ranges of values. For example, the reference value can be derived from the average gen copy number and/or expression values obtained from a group of healthy control subjects or non-tumor tissue from a group of cancer patients.

In additional examples, the control is a threshold value. A threshold level of EPS8L1 is a quantified level of EPS8L1 nucleic acid (such as EPS8L1 mRNA or EPS8L1 gene copy number) or EPS8L1 protein. An amount of EPS8L1 nucleic acid or protein in a sample that exceeds the threshold level is predictive of a particular disease state or outcome (such as a poor prognosis) in a subject with a breast tumor. The nature and numerical value (if any) of a threshold level will vary based on the method chosen to determine the amount of EPS8L1 nucleic acid. One of skill in the art can determine a threshold level of EPS8L1 nucleic acid or protein in a sample that would be predictive of reduced survival using any method of measuring amounts of nucleic acid or protein now known in the art or yet to be disclosed.

In some examples, a threshold level of EPS8L1 includes multiple threshold levels, such as high, medium, or low probability of survival (for example, overall survival). In other examples, there could be a low threshold amount wherein an amount of EPS8L1 in the sample below the threshold indicates that the subject is likely to have a good prognosis and a separate high threshold amount above which an amount of EPS8L1 indicates that the subject has a poor prognosis. An amount of EPS8L1 between the two threshold values is considered inconclusive as to prognosis of the subject. In some examples, multiple thresholds are selected by so-called “tertile,” “quartile,” or “quintile” analyses. In these methods, multiple groups are considered together as a single population, and are divided into 3 or more “bins” having equal numbers of individuals. The boundary between two of these bins may be considered a threshold level indicating a particular level of risk that the subject has or will have a poor prognosis. A risk may be assigned based on which bin a test subject falls into.

To obtain a threshold value of EPS8L1 nucleic acid or protein that indicates that a subject has a poor prognosis for a particular method of measuring EPS8L1 (for example, RT-PCR, ELISA, ISH, or IHC) an EPS8L1 amount is determined using samples obtained from a first cohort of subjects with a breast tumor known to have a poor prognosis and from a second cohort known to have a good prognosis. In one exemplary embodiment, the first cohort includes subjects with survival for less than 50 months and the second cohort includes subjects with survival for more than 50 months. However, one of ordinary skill in the art can select different cohorts that are appropriate for determining a threshold value. EPS8L1 nucleic acid or protein is determined in both cohorts and an amount of EPS8L1 that signifies that a subject has a poor prognosis is determined to be a threshold value. In some examples, the threshold is the amount of EPS8L1 nucleic acid or protein that provides the maximal ability to predict poor prognosis and maximizes both the selectivity and sensitivity of the test. The predictive power a threshold level of expression may be evaluated by any of a number of statistical methods known in the art (such as receiver operating characteristic area under the curve (ROC AUC), odds ratio, or hazard ratio).

In particular embodiments, the disclosed methods further include administering a treatment to the subject with the breast tumor. Increased EPS8L1 has been found to be particularly predictive in ErbB2-positive tumors whether or not those tumors are ER-positive or ER-negative (see Example 4, below). Therefore, in some examples, a subject identified as having poor prognosis using the methods disclosed herein (for example, having a breast tumor with increased EPS8L1 nucleic acid or protein) is administered an ErbB2 (Her2)-targeting therapy. ErbB2-targeting therapies include trastuzumab, lapatinib, pertuzumab, or combinations thereof. One of ordinary skill in the art can select additional ErbB2-targeting therapies, including those now known or developed in the future. In some examples, the subject is administered a combination of an ErbB2-targeting therapy and an anti-estrogen therapy. In other examples, a subject identified as having a good prognosis using the methods disclosed herein (for example, having a breast tumor with normal or decreased EPS8L1 nucleic acid or protein) is treated with standard care (such as surgery, radiation, and/or neo-adjuvant chemotherapy).

In additional examples, the subject may also be administered one or more anti-hormone therapies, such as tamoxifen, letrozole, toremifene, fulvestrant, anastrozole, exemestane, or combinations thereof. The subject may also be administered one or more adjuvant chemotherapeutics, such as taxanes (such as paclitaxel or docetaxel), anthracyclines (such as daunorubicin, doxorubicin, epirubicin, or mitoxantrone), cyclophosphamide, capecitabine, 5-fluorouracil, methotrexate, or combinations thereof. In still further examples, a subject with increased EPS8L1 nucleic acid or protein may be treated surgically, for example by removing additional tissue, taking wider tumor margins, and/or removing additional lymph nodes. The subject may also be treated with radiation therapy.

### III. METHODS OF DETECTING NUCLEIC ACID OR PROTEIN

As described below, an amount of a nucleic acid or protein (such as EPS8L1 or ErbB2) in a sample can be detected using any one of a number of methods well known in the art. Although exemplary methods are provided, the disclosure is not limited to such methods. Furthermore, although the methods below are described with specific reference to EPS8L1, one of ordinary skill in the art would understand that similar methods could be utilized to detect other nucleic acids or proteins of interest (including, but not limited to ErbB2).

A. Methods for Detecting mRNA or cDNA

Gene expression can be evaluated by detecting mRNA (or cDNA) encoding a protein, such as EPS8L1. In some examples, the mRNA (or cDNA) is quantitated. RNA can be isolated from a sample from a subject (such as a tumor sample from a subject with a breast tumor, a sample of adjacent non-tumor tissue from the subject, a sample of tumor-free tissue from a normal (healthy) subject, or combinations thereof), using methods well known to one skilled in the art, including commercially available kits. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Biotechniques 6:56-60 (1988), and De Andres et al., Biotechniques 18:42-44 (1995). In one example, RNA isolation can be performed using purification kit, buffer set and protease from commercial manufacturers, such as RNeasy® mini-columns (Qiagen, Valencia, Calif.), MASTERPURE® Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from tumor or other biological sample can be isolated, for example, by cesium chloride density gradient centrifugation.

Methods of determining EPS8L1 mRNA (e.g., EPS8L1 gene expression) include methods based on hybridization analysis of polynucleotides, methods based on sequencing of polynucleotides, and proteomics-based methods. In some examples, mRNA expression in a sample is quantified using Northern blotting or in situ hybridization (Parker & Barnes, Methods in Molecular Biology 106:247-283, 1999); RNAse protection assays (Hod, Biotechniques 13:852-4, 1992); or PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

In some examples, EPS8L1 mRNA is detected with RT-PCR or real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are commercially available, for example from PE Applied Biosystems (Foster City, Calif.).

In some examples, EPS8L1 expression is identified or confirmed using a microarray. The EPS8L1 mRNA (or cDNA) can be measured in either fresh or paraffin-embedded tumor tissue, using microarray technology. In this method, an array includes one or more probes for EPS8L1. The array is then hybridized with isolated nucleic acids (such as cDNA or mRNA) from a sample. The microarray may also include one or more control probes, such as probes for one or more housekeeping genes.

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to qualitatively or semi-quantitatively assess EPS8L1 mRNA expression in a breast tumor sample, such as a FFPE breast tumor sample or a tumor microarray.

In some embodiments of the detection methods, the expression of one or more “housekeeping” genes or “internal controls” can also be evaluated. These terms include any constitutively or globally expressed gene (or protein, as discussed below) whose presence enables an assessment of EPS8L1 mRNA, cDNA or protein levels. Such an assessment includes a determination of the overall constitutive level of gene transcription and a control for variations in RNA (or protein) recovery.

B. Methods for Determining Gene Copy Number

In some examples of the disclosed methods, gene copy number (such as EPS8L1 gene copy number or gene amplification) is determined. In some examples, the methods include in situ hybridization (such as fluorescent, chromogenic, or silver in situ hybridization), comparative genomic hybridization, or polymerase chain reaction (such as real-time quantitative PCR).

In particular examples, gene copy number is determined by in situ hybridization (ISH), such as fluorescence in situ hybridization (FISH), chromogenic in situ hybridization (CISH), or silver in situ hybridization (SISH). In ISH methods, a sample is contacted with an EPS8L1 genomic DNA probe and hybridization of the probe to chromosomes or nuclei in the sample is detected directly or indirectly. For example, using FISH, a DNA probe (such as an EPS8L1 probe) is labeled with a fluorescent dye or a hapten. Hybridization of the probe to chromosomes or nuclei is visualized either directly (in the case of a fluor-labeled probe) or indirectly (using fluorescently labeled anti-hapten antibodies to detect a hapten-labeled probe). For CISH, the probe is labeled with a hapten (such as digoxigenin, biotin, or fluorescein) and is detected with an anti-hapten antibody, which is either conjugated to an enzyme (such as horseradish peroxidase or alkaline phosphatase) that produces a colored product at the site of the hybridized probe in the presence of an appropriate substrate (such as DAB, NBT/BCIP, etc.), or with a secondary antibody conjugated to the enzyme. Similarly, in SISH a hapten-labeled probe is detected with an anti-hapten antibody, except that the enzyme (such as horseradish peroxidase) conjugated to the antibody (either anti-hapten antibody or a secondary antibody) catalyzes deposition of metal nanoparticles (such as silver or gold) at the site of the hybridized probe. EPS8L1 copy number may be determined by counting the number of fluorescent, colored, or silver spots on the chromosome or nucleus. The number of copies of the gene (or chromosome) may be estimated by a person of skill in the art, such as a pathologist or computer, in the case of an automated method.

In other examples, both the EPS8L1 gene and Chromosome 19 DNA (such as Chromosome 19 centromeric DNA) are detected in a sample from the subject, for example by ISH. EPS8L1 and Chromosome 19 copy number may be determined by counting the number of fluorescent, colored, or silver spots on the chromosome or nucleus. A ratio of EPS8L1 gene copy number to Chromosome 19 number is then determined. Chromosome 19 centromeric probes are commercially available, for example, SureFISH Chr 19 CEP (Agilent Technologies, Santa Clara, Calif.) or SE 1/15/19 satellite enumeration probe (Kreatech Diagnostics, Amsterdam, The Netherlands).

In other examples, comparative genomic hybridization (CGH) is used to determine EPS8L1 gene copy number. See, e.g., Kallioniemi et al., Science 258:818-821, 1992; U.S. Pat. Nos. 5,665,549 and 5,721,098. In one example, DNA from a tumor sample and from control tissue (reference, such as a non-breast tumor sample) are labeled with different detectable labels. The tumor and reference DNA samples are mixed and the mix is hybridized to normal metaphase chromosomes. The fluorescence intensity ratio along the chromosomes is used to evaluate regions of DNA gain or loss in the tumor sample.

EPS8L1 gene copy number may also be determined by array CGH (aCGH). See, e.g., Pinkel and Albertson, Nat. Genet. 37:S11-S17, 2005; Pinkel et al., Nat. Genet. 20:207-211, 1998; Pollack et al., Nat. Genet. 23:41-46, 1999. aCGH is similar to standard CGH, however, for aCGH, the DNA mixture is hybridized to a slide containing tens, hundreds, or thousands of defined DNA probes (such as probes that are homologous to portions of the EPS8L1 gene). The fluorescence intensity ratio at each probe in the array is used to evaluate regions of DNA gain or loss in the tumor sample, which can be mapped in finer detail than CGH, based on the particular probes which exhibit altered fluorescence intensity.

In another example, EPS8L1 copy number is determined by real-time quantitative PCR (RT-qPCR), such as with a TAQMAN assay. See, e.g., U.S. Pat. No. 6,180,349. The EPS8L1 copy number is determined relative to a normalization gene contained within the sample, which has a known copy number (see Heid et al., Genome Research 6:986-994, 1996). Quantitative PCR is also described in U.S. Pat. No. 5,538,848.

Additional methods that may be used to determine EPS8L1 gene copy number are known to those of skill in the art. These methods include, but are not limited to Southern blotting, multiplex ligation-dependent probe amplification (MLPA; see, e.g., Schouten et al., Nucl. Acids Res. 30:e57, 2002), and high-density SNP genotyping arrays (see, e.g. WO 98/030883).

C. Methods for Detecting Protein

In some examples, expression of protein (such as EPS8L1 protein) is analyzed. Suitable biological samples include samples containing protein obtained from a tumor (such as a breast tumor) of a subject, from non-tumor tissue of the subject, and/or protein obtained from one or more samples of cancer-free subjects.

Antibodies specific for EPS8L1 can be used for detection and quantitation of EPS8L1 protein by one of a number of immunoassay methods that are well known in the art, such as those presented in Harlow and Lane (Antibodies, A Laboratory Manual, CSHL, New York, 1988). Methods of constructing such antibodies are known in the art. In addition, such antibodies may be commercially available.

Exemplary commercially available EPS8L1 antibodies include anti-EPS8L1 antibodies from Abcam (Cambridge, Mass., for example, catalog numbers ab58687, ab64839, ab129547, and ab169701), Santa Cruz Biotechnology (Santa Cruz, Calif., for example, catalog numbers sc-132673, sc-132672, and sc-101950), and Abnova (Walnut, Calif., for example, catalog numbers H00054869-B01, H00054869-B01P, and H00054869-A01).

Any standard immunoassay format (such as ELISA, Western blot, or RIA assay) can be used to measure EPS8L1 protein levels. Immunohistochemical techniques can also be utilized for EPS8L1 protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantitating EPS8L1 protein, a biological sample of the subject that includes proteins (such as a breast tumor sample) can be used. The amount of EPS8L1 protein can be assessed in the sample, and optionally in adjacent non-tumor tissue in a tumor sample, or in tissue from cancer-free subjects. The amount of EPS8L1 protein in the sample can be compared to levels of the protein found in cells from a cancer-free subject or other control (such as a standard value or reference value). A significant increase or decrease in the amount can be evaluated using statistical methods known in the art.

In an additional example, EPS8L1 protein can be detected in a sample using an electrochemical immunoassay method. See, e.g., Yu et al., J. Am. Chem. Soc. 128:11199-11205, 2006; Mani et al., ACS Nano 3:585-594, 2009; Malhotra et al., Anal. Chem. 82:3118-3123, 2010. In this method, an antibody (such as an anti-EPS8L1 antibody) is conjugated to terminally carboxylated single-wall carbon nanotubes (SWNT), multi-wall carbon nanotubes (MWCNT), or gold nanoparticles (AuNP), which are attached to a conductive surface. The SWNTs, MWCNTs, or AuNPs, are contacted with a sample and EPS8L1 protein in the sample binds to the primary antibody. A second antibody conjugated directly or indirectly to a redox enzyme (such as horseradish peroxidase) binds to the primary antibody or to EPS8L1 protein (for example, in a “sandwich” assay). Signals are generated by adding enzyme substrate (e.g. hydrogen peroxide if the enzyme is HRP) to the solution bathing the sensor and measuring the current produced by the catalytic reduction.

Quantitative spectroscopic methods, such as SELDI, can be used to analyze EPS8L1 protein expression in a sample (such as tumor tissue, non-cancerous tissue, and tissue from a cancer-free subject). In one example, surface-enhanced laser desorption-ionization time-of-flight (SELDI-TOF) mass spectrometry is used to detect protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

### EXAMPLES

The following examples are illustrative of disclosed methods. In light of this disclosure, those of skill in the art will recognize that variations of these examples and other examples of the disclosed methods would be possible without undue experimentation.

### Example 1

### Identification of Growth Inhibitory and Apoptosis Inducing RNAi Targets with ERBB2 Subset Specificity Across ERBB2 Positive Breast Cancer Lines

To identify genes essential to growth and survival of the Her2 positive (ErbB2 positive) subgroup of breast cancer cells, a set of siRNA screens using the cell spot microarray (CSMA) RNAi platform (Rantala et al, BMC Genomics 12:162 (2011); incorporated by reference herein) was performed. With the presumption that tumors within the same breast cancer subtype would share molecular mechanisms for their growth (proliferation and/or survival), a custom siRNA library was developed that included genes most frequently amplified in clinical Her2 positive subgroup of breast cancers according to the Cancer Genome Atlas network summary for breast cancer (The Cancer Genome Atlas Network, Nature 490:61-70, 2012, incorporated by reference herein).

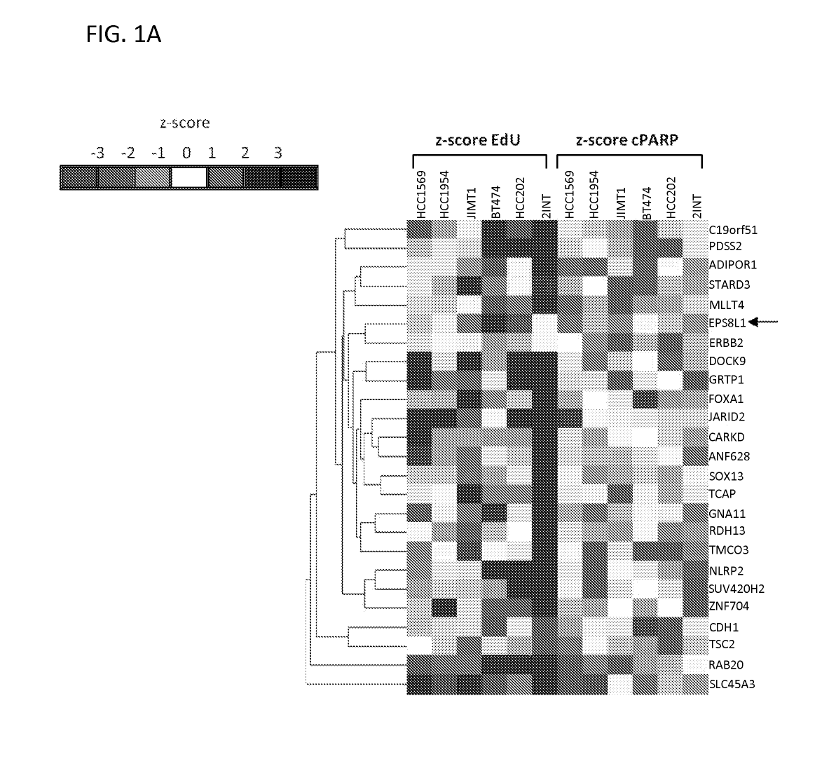

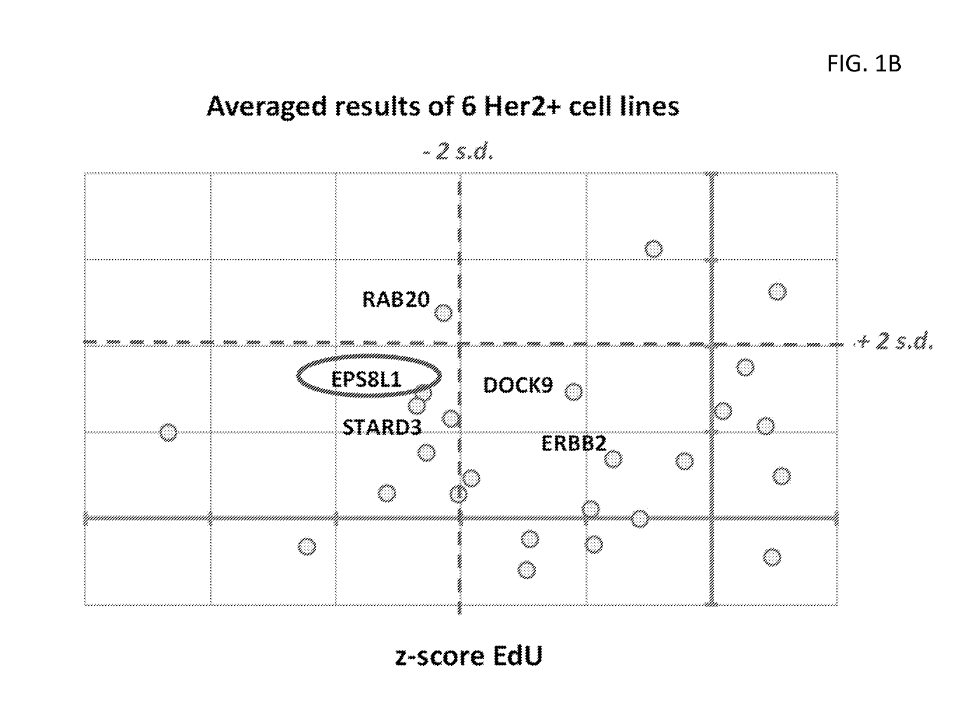

A total of six cell lines were selected for the screening experiments based on their known genomic subtype. These six cell lines were HCC1569, BT474, 21NT, JIMT1, HCC202 and HCC1954. A single functionally validated siRNA was selected for each gene when available and two siRNAs were selected for each gene for which no pre-validated siRNA was available. Negative control siRNAs with a non-targeting sequence (Qiagen AllStar® Negative Control) were used as negative transfection controls. After 72 hours of transfection on the CSMAs, an antibody based detection of cleaved PARP and a fluorescence detection assay for EdU incorporation (Invitrogen) were used to assess cell growth and induction of apoptosis as consequence of the RNAi gene silencing. The RNAi targeted genes were considered as Her2 positive subgroup specific growth promoting genes when their down-regulation caused an average reduction of proliferation of more than two standard deviations (z-score <−2) or an increase of cPARP signal of more than two standard deviations (z-score >2) across all the tested cell lines (FIG. 1A). The RNAi of EPS8L1 that is an siRNA targeting the sequence AACAGCCTCCGTGCTTAGCA (SEQ ID NO: 1; Qiagen, Hs_EPS8L1_14) was identified among the strongest growth inhibitory siRNAs across all 6 Her2 positive breast cancer cell lines (FIG. 1B).

### Example 2

### Analysis of Genomic Aberrations and Expression Patterns of EPS8L1 in Breast Cancer

Analysis of genomic aberrations of EPS8L1 and expression patterns across clinical cancer samples and breast cancer cell lines was performed based on publicly available datasets from The Cancer Genome Atlas (TCGA, available on the World Wide Web at cbioportal.org/public-portal) and supplementary data (Neve et al, Cancer Cell 10, 515-527 (2006); incorporated by reference herein), respectively.

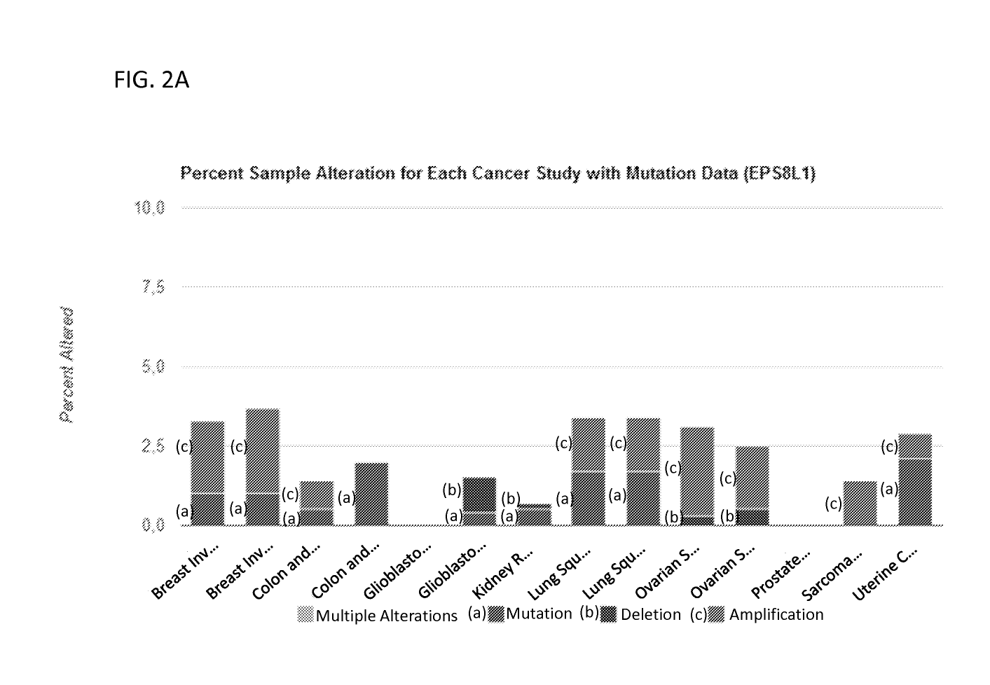

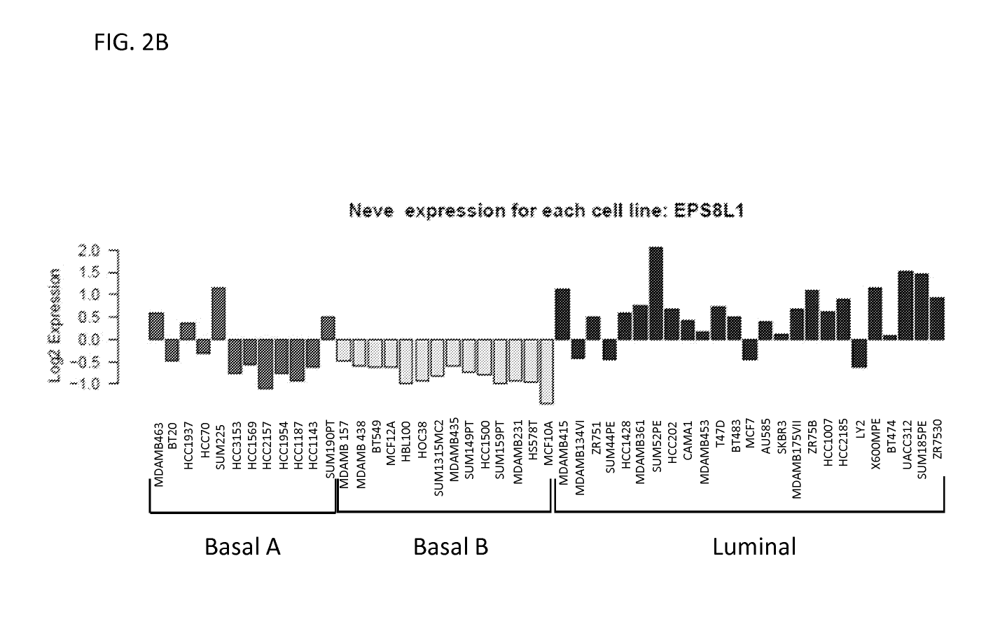

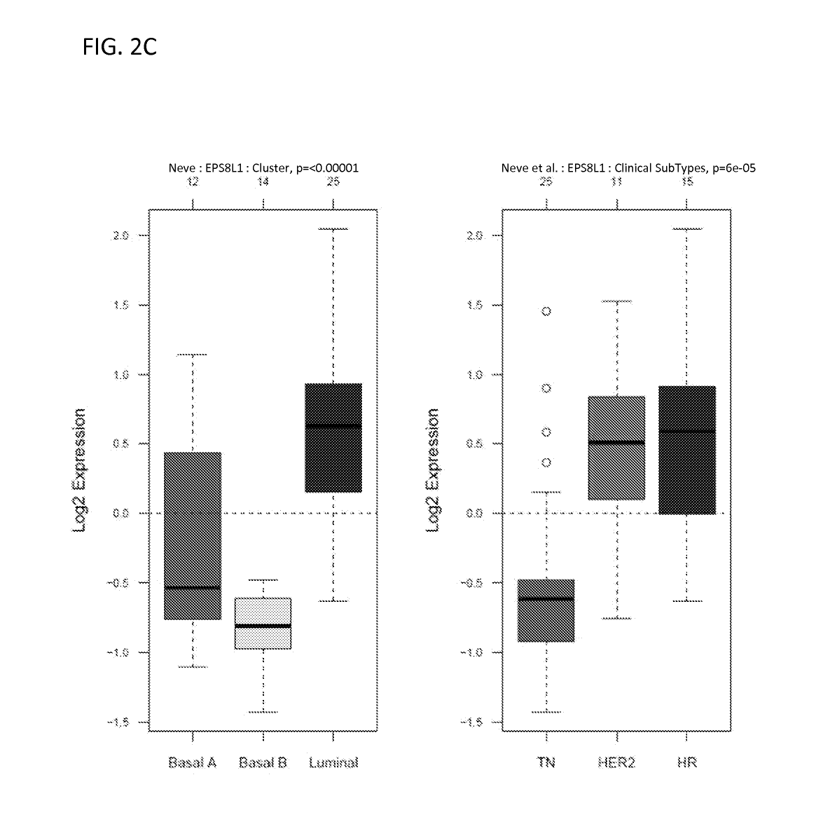

FIG. 2A indicates the frequency of EPS8L1 aberrations across solid tumors across the TCGA data. FIG. 2B shows the median centered expression of EPS8L1 across 51 individual breast cancer cell lines analyzed using an Affymetrix U133A® platform and processed as previously described (Staaf et al., 2010. J. Clin. Oncol. 28: 1813-1820, which is incorporated by reference herein). Cell lines are grouped in the basal A (red, left), basal B (grey, center) and luminal (blue, right) subgroups (Neve et al.). FIG. 2C displays expression for EPS8L1 in the 51 cell lines of FIG. 2B grouped into clinical subtypes; triple negative (TN), HER2-positive (HER2), and hormone receptor positive (HR) based on annotation data from Neve et al. This stratification of EPS8L1 expression in model cell lines of human breast cancers indicates EPS8L1 to be most highly expressed in cells of luminal origin, whether separated according to ESR1 (estrogen receptor alpha) expression status or ErbB2 amplification and/or overexpression status.

### Example 3

### Effects of Silencing of EPS8L1 Expression on Cell Growth and Viability

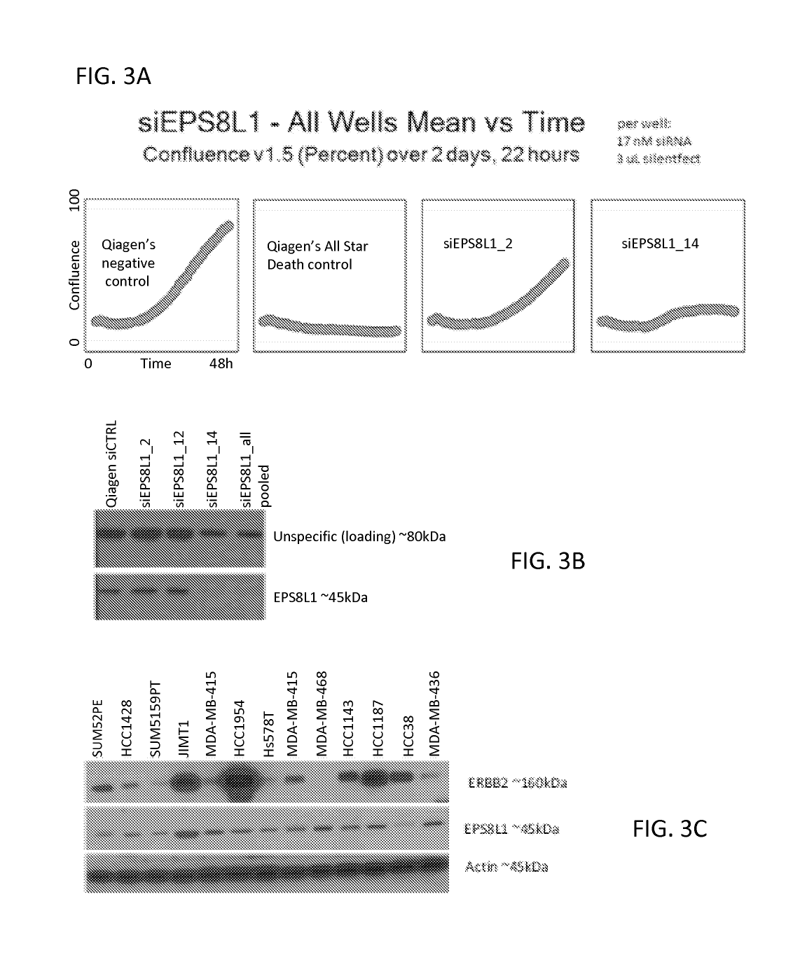

Validation experiments with siRNAs for EPS8L1 cell growth and viability effects were performed with live cell imaging. EPS8L1 siRNAs (Qiagen, Hs_EPS8L1_2 and Hs_EPS8L1_14) were contacted with JIMT1 breast cancer cells and the growth measured. Cells were cultured on clear bottom 96-well plates, (10,000 cells per well) and 12-well (2×10⁵ cells per well) plates and transfected with the 17 nM siRNA constructs (Qiagen) using siLentFect® (Bio-Rad) in ratio of 1:600 (v/v). Time-lapse imaging of the transfected cells was performed with an Incucyte® HD live-cell imaging microscope using a 20x objective (Essen Instruments, Ann Arbor, Mich.). Images were acquired every 2 hours for 2 days. Comparative analysis of well confluence as a measure of cell growth indicated that transfecting cells with the EPS8L1 siRNA Hs_EPS8L1_14 resulted in over 80% growth inhibition in comparison to non-targeting control, while siRNA Hs_EPS8L1_2 resulted in 15% growth inhibition in comparison to non-targeting siRNA transfection control (FIG. 3A).

Western blots confirming that EPS8L1 siRNAs inhibit EPS8L1 protein expression are shown in FIG. 3B and FIG. 3C. Total cell lysates were fractionated on SDS-polyacrylamide gels and transferred to nitrocellulose membranes (Whatman Inc). The filters were blocked against non-specific binding using 5% skim milk. Membranes were probed with antibodies overnight at 4° C. (EPS8L1; 1:1000, Abcam, Cambridge, Mass., cat. no. Ab58687). Equal loading was confirmed by probing the same filter with a non-specific antibody for tubulin (1:5000, Abcam). Signals were revealed by incubating the filters with horseradish peroxidase-coupled goat anti-mouse IgG secondary antibody and goat anti-rabbit antibody (1:1000; Sigma). FIG. 3B shows silencing of EPS8L1 by EPS8L1 specific siRNA. FIG. 3C shows EPS8L1 silencing by EPS8L1 specific siRNA in the indicated human breast cancer cell lines. Beta-Actin was used as a loading control.

### Example 4

### High EPS8L Expression in Subgroups of Clinical Breast Cancer Samples is Predictive of Poor Overall Survival

A data set including gene expression data and annotation data for a pooled 1881-sample breast tumor set from publicly available sources was used to assess the clinical relevance of EPS8L1 expression. The 1881-sample breast tumor set comprises 11 public data sets analyzed using Affymetrix U133A arrays (Table 1).

TABLE 1
1881-sample breast tumor set summary

| | Number of | | | DMFS | Mean DMFS | | Mean OS |
|---|---|---|---|---|---|---|---|
| GEO ID | samples | ER−/+ | LM−/+ | (0/1) | (years) | OS (0/1) | (years) |
| GSE7390 | 198 | 64/134 | 198/0 | 136/62 | 10.8 +/− 5.4 | 142/56 | 11.463.7 |
| GSE3494 | 251 | 34/213 | 158/84 | NA | NA | 132/119 | 7.964.1 |
| GSE1456 | 159 | 29/130 | 94/60 | NA | NA | 119/40 | 6.461.9 |
| GSE2034 | 286 | 77/209 | 286/0 | 179/107 | 6.5 +/− 3.5 | NA | NA |
| GSE2603 | 99 | 42/57 | 34/65 | 55/27 | 5.2 +/− 2.3 | NA | NA |
| GSE6532 | 327 | 45/262 | 221/85 | 225/68 | 6.3 +/− 3.7 | NA | NA |
| GSE4922 | 40 | NA | NA | NA | NA | NA | NA |
| GSE12093 | 136 | 0/136 | 136/0 | 116/20 | 7.7 +/− 3.2 | NA | NA |
| GSE5327 | 58 | 58/0 | NA | 47/11 | 6.8 +/− 3.1 | NA | NA |
| GSE11121 | 197 | NA | 197/0 | 153/44 | 7.8 +/− 4.2 | NA | NA |
| Chin | 130 | 46/84 | 59/71 | 102/27 | 5.7 +/− 4 | 84/45 | 6.463.7 |
| Total | 1881 | 395/1225 | 1383/365 | 1013/366 | 7.2 +/− 4.2 | 477/260 | 8.264.4 |

| | | Mean RFS | | Median age | Mean size |
|---|---|---|---|---|---|
| GEO ID | RFS | (years) | Grade: 1/2/3 | (years) | (mm) |
| GSE7390 | 107/91 | 9.365.6 | 30/83/83 | 4667 | 22 +/− 8 |
| GSE3494 | 155/96 | 5.563.4 | 67/128/54 | 64614 | 22 +/− 13 |
| GSE1456 | 119/40 | 6.262.3 | 28/58/61 | 56614 | 22 +/− 12 |
| GSE2034 | NA | NA | 6/42/139* | 53612* | 10 +/− 6 |
| GSE2603 | NA | NA | NA | 56614 | 3 +/−+/− 17 |
| GSE6532 | 195/111 | 6.363.7 | 65/145/60 | 60.5612 | 23 +/− 12 |
| GSE4922 | NA | NA | 0/40/0 | NA | NA |
| GSE12093 | NA | NA | NA | NA | NA |
| GSE5327 | NA | NA | NA | NA | NA |
| GSE11121 | NA | NA | 29/135/33 | NA | 21 +/− 10 |
| Chin | NA | NA | 14/46/65 | 51615 | 27 +/− 14 |
| Total | 576/338 | 6.764.2 | 239/677/495 | 55613 | 20 +/− 12 |

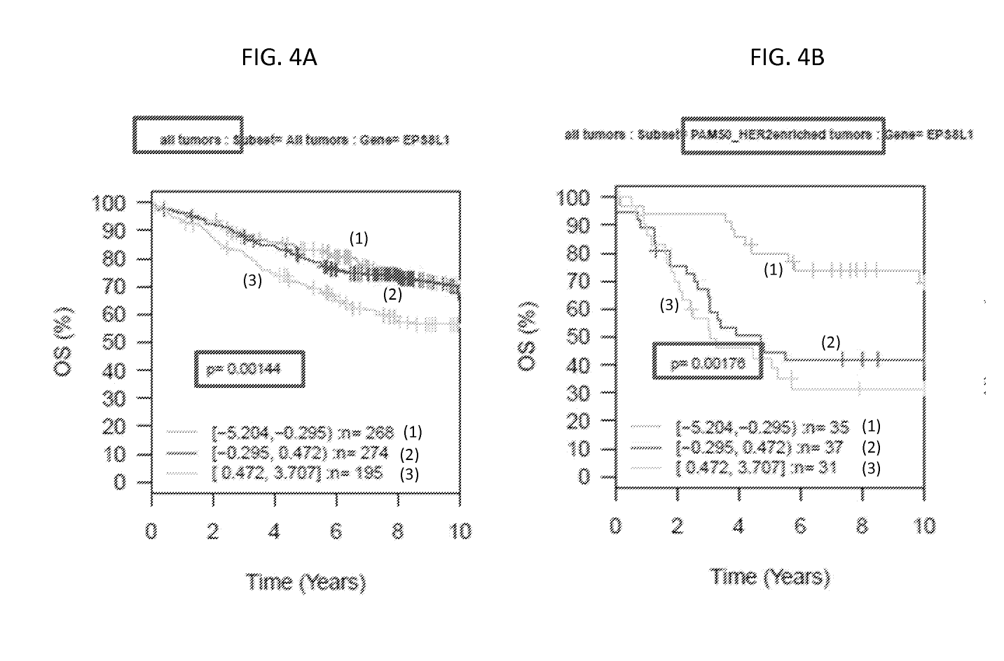

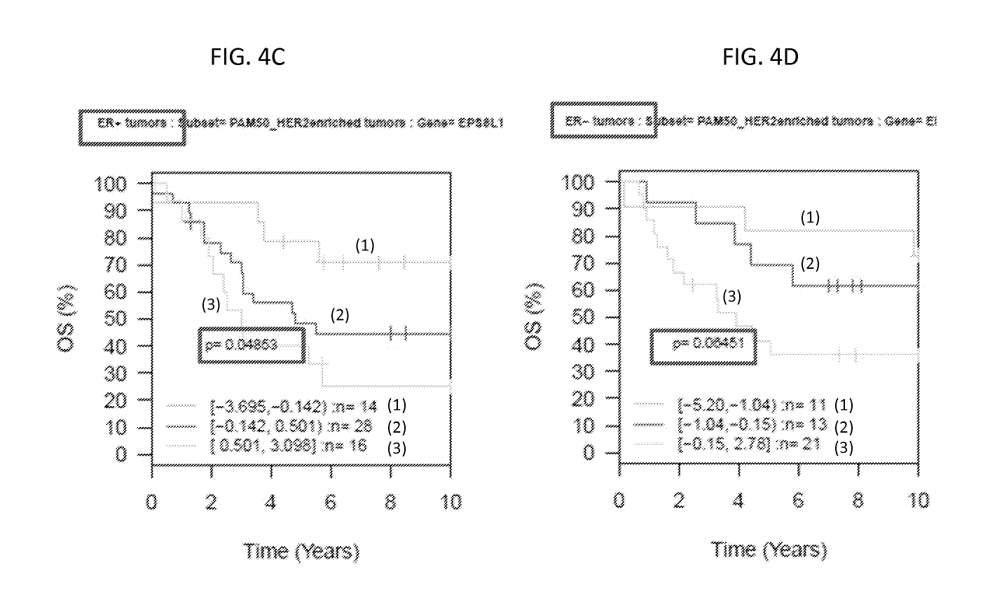

Association of EPS8L1 gene expression levels with outcome was performed using overall survival (OS) as endpoint and 10-year censoring. Samples in the 1881-sample set were stratified into three quantiles based on EPS8L1 expression level (log 2 expression): EPS8L1_low (expression in a range of −5.204 to −0.295 relative to median 0); EPS8L1_medium (expression from −0.295-0.472 relative to median 0); and EPS8L1_high (expression from 0.472-3.707 relative to median 0), followed by Kaplan-Meier survival analysis in the subgroups (Ringner et al., PLoS One 6:e17911, 2011, incorporated by reference herien). Log-rank P-values are shown as −log 10 (P-value). FIGS. 4A-4D show that EPS8L1 expression is predictive of overall survival in all tumors with EPS8L1_high expression and that it is particularly predictive in HER2-positive tumors whether or not those tumors are ER-positive or ER-negative (FIG. 4B-4D).

### Example 5

### High EPS8L1 Copy Number in Genomic DNA is Predictive of Poor Overall Survival in Breast Cancer Patients

Copy number changes for EPS8L1 from 178 primary breast cancer cases were extracted from Hu-244A CGH microarrays (Agilent Technologies). The tumors are part of a cohort of 212 primary breast cancer cases sequentially collected at Oslo University Hospital Ullevål, Norway, from 1990 to 1994 with an observation time of 12 to 16 years (Langerød A et al. 2009 Breast Cancer Res. 9(3):R30; incorporated by reference herein). The samples were profiled by standard protocol (Barrett M T et al. 2004 Proc Natl Acad Sci USA 101(51):17765-17770; incorporated by reference herein) without a prelabeling amplification step. Scanned microarray images were read and analyzed with Feature Extraction® v9.5 (Agilent Technologies) with protocols (CGHv4_95_Feb07 and CGH-v4 91 2) for aCGH preprocessing, which included linear normalization. Data were segmented using the PCF (Piecewise Constant Fit) algorithm with settings K min=5 and γ=25. Aberrations were scored with a threshold of 0.3; gain >0.3 and loss <−0.3 (log 2 scale in comparison to gene signals of chromosome 19). Statistical association of copy number changes for EPS8L1 and survival were performed in SPSS 16.0 (SPSS, Inc, Chicago, Ill.). The ASCO guideline for defining Erb B2 amplification using FISH (ratio of ErbB22 gene signals to chromosome 17 signals of more than 2.2; Wolff et al., Arch. Pathol. Lab. Med. 131:18-43, 2007) was used to define tumors to be ErbB2 and EPS8L1 amplified with copy number gain >0.9 (log 2 scale).

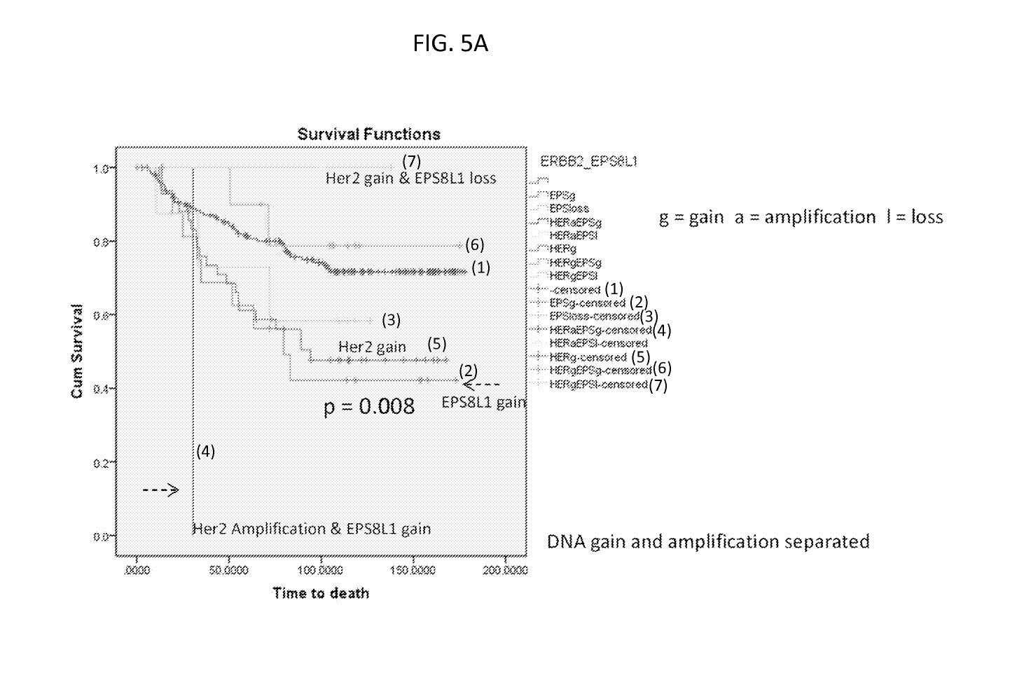

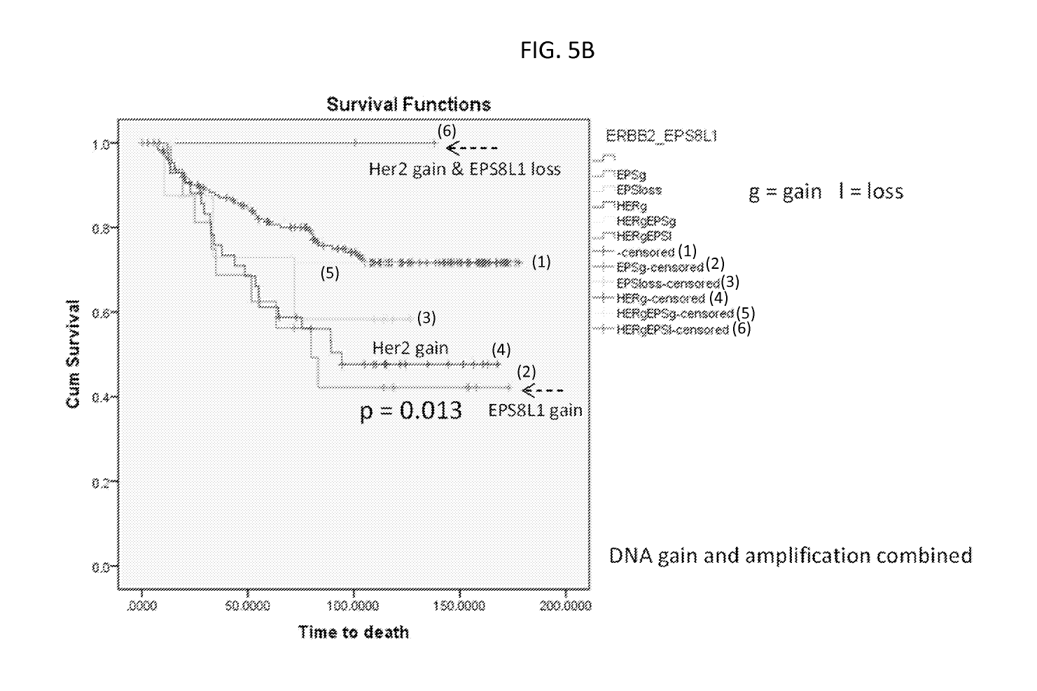

FIGS. 5A and B are a set of two plots showing Kaplan-Meier analysis of overall survival times based on EPS8L1 copy number. FIG. 5A has DNA gain and amplification separated, while FIG. 5B has DNA gain and amplification combined. Subjects with EPS8L1 gain had decreased survival time (FIGS. 5A and B) and subjects with both ErbB2 (HER2) amplification and EPS8L1 loss had especially poor cumulative survival time (FIG. 5B). Interestingly, subjects with ErbB2 (HER2) gain and EPS8L1 loss had particularly good survival (FIGS. 5A and B), and could even have benign or very slowly progressing tumors. These results indicate the applicability of detection of EPS8L1 DNA copy number alterations for clinical prognosis as a single marker or in combination with detection of ErbB2 copy number status.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of determining prognosis of a subject with a breast tumor, comprising:
determining an amount of an EPS8-like 1 (EPS8L1) nucleic acid or protein in a sample from the subject;
comparing the amount of the EPS8L1 nucleic acid or protein to a control; and
determining that the subject has a poor prognosis if the amount of the EPS8L1 nucleic acid or protein is increased compared to the control.

2. The method of claim 1, wherein determining the amount of the EPS8L1 nucleic acid or protein comprises determining the amount of an EPS8L1 nucleic acid and the amount of the EPS8L1 nucleic acid comprises an amount of genomic DNA, cDNA, or mRNA, determining EPS8L1 gene copy number, or determining the amount of an EPS8L1 protein.

3. The method of claim 2, wherein determining the amount of the EPS8L1 nucleic acid comprises one or more of microarray analysis, polymerase chain reaction (PCR), reverse transcription PCR, real-time reverse transcription PCR, in situ hybridization, nuclease protection, and comparative genomic hybridization or wherein determining the amount of the EPS8L1 protein comprises an immunoassay.

4-6. (canceled)

7. The method of claim 3, wherein the immunoassay is Western blotting, immunohistochemistry, ELISA, or electrochemical immunoassay.

8. The method of claim 1, wherein the poor prognosis comprises decreased overall survival, decreased relapse-free survival, or decreased metastasis-free survival.

9. The method of claim 8, wherein the decreased overall survival comprises decreased survival over 10 years, over 5 years, or over 3 years.

10-11. (canceled)

12. The method of claim 1, wherein the control is a threshold level of EPS8L1 nucleic acid or protein.

13. The method of claim 1, further comprising:
determining an amount of an ErbB2 nucleic acid or protein in the sample; and
comparing the amount of the ErbB2 nucleic acid or protein to a control, wherein an increased amount of ErbB2 nucleic acid or protein compared to the control indicates that the subject has a poor prognosis.

14. The method of claim 1, wherein the sample is a breast tumor sample from the subject.

15. The method of claim 14, wherein the tumor sample from the subject comprises a tissue biopsy or a fine needle aspirate.

16. The method of claim 15, wherein the tissue biopsy comprises a tissue section.

17. The method of claim 14, wherein the tumor sample from the subject comprises a fresh tumor sample, a frozen tumor sample, or a fixed tumor sample.

18. The method of claim 1, further comprising obtaining the sample from the subject.

19. The method of claim 1, further comprising administering a therapeutic agent to the subject.

20. The method of claim 19, wherein the therapeutic agent comprises an ErbB2-specific agent.

21. The method of claim 20, wherein the ErbB2-specific agent comprises trastuzumab or lapatinib.

22. An in vitro method of determining prognosis of a subject with a breast tumor, comprising:
(i) determining an amount of EPS8-like 1 (EPS8L1) nucleic acid or protein in a sample from the subject; and
comparing the amount of the EPS8L1 nucleic acid or protein to a control;
(ii) determining an amount of ErbB2 nucleic acid or protein in the sample from the subject; and
comparing the amount of the ErbB2 nucleic acid or protein to a control; and
(iii) determining that the subject has a poor prognosis if the amount of the EPS8L1 nucleic acid or protein is increased compared to the control and the amount of the ErbB2 nucleic acid or protein is increased compared to the control.

23. The method of claim 22, wherein determining the amount of the EPS8L1 and/or ErbB2 nucleic acid or protein comprises:
determining the amount of an EPS8L1 and/or ErbB2 nucleic acid and the amount of the EPS8L1 and/or ErbB2 nucleic acid comprises an amount of genomic DNA, cDNA, or mRNA;
determining the amount of the EPS8L1 nucleic acid comprises determining EPS8L1 gene copy number and/or determining the amount of the ErbB2 nucleic acid comprises determining ErbB2 gene copy number; and/or
determining the amount of the EPS8L1 and/or ErbB2 nucleic acid or protein comprises determining the amount of an EPS8L1 and/or ErbB2 protein.

24. The method of claim 23, wherein determining the amount of the EPS8L1 and/or ErbB2 nucleic acid comprises one or more of microarray analysis, polymerase chain reaction (PCR), reverse transcription PCR, real-time reverse transcription PCR, in situ hybridization, nuclease protection, and comparative genomic hybridization or wherein determining the amount of the EPS8L1 and/or ErbB2 protein comprises an immunoassay.

25-27. (canceled)

28. The method of claim 24, wherein the immunoassay is Western blotting, immunohistochemistry, ELISA, or electrochemical immunoassay.

29. The method of claim 22, wherein the poor prognosis comprises decreased overall survival, decreased relapse-free survival, or decreased metastasis-free survival.

30. The method of claim 22, wherein the sample is a breast tumor sample from the subject.

31. The method of claim 22, further comprising administering a therapeutic agent to the subject.

32. The method of claim 31, wherein the therapeutic agent comprises an ErbB2-specific agent.
